(19) United States
(12) Patent Application Publication
(10) Pub. No.: US 2010/0166603 A1
(43) Pub. Date: Jul. 1, 2010

(54) Powder Sterilization

(75) Inventor: David OPIE, Baltimore, MD (US)

Correspondence Address: PILLSBURY WINTHROP SHAW PITTMAN, LLP, P.O. BOX 10500, MCLEAN, VA, 22102, US

(73) Assignee: NOXILIZER, Inc., Bethesda, MD (US)

(21) Appl. No.: 12/608,750

(22) Filed: Oct. 29, 2009

Related U.S. Application Data
(60) Provisional application No. 61/110,280, filed on Oct. 31, 2008.

(51) Int. Cl. A61L 2/20; A61L 2/26

(52) U.S. Cl. 422/32; 422/300; 422/292; 422/293; 422/302

(57) ABSTRACT

A system for sterilizing a powder includes a device for agitating the powder during application of a sterilizing gas including nitrogen dioxide and humidity. A related method includes agitating the powder while applying the sterilizing gas.

### RELATED APPLICATION

This application claims priority to U.S. Provisional Patent Application No. 61/110,280 filed Oct. 31, 2008, which is incorporated herein by reference in its entirety.

### BACKGROUND

1. Field of the Invention

The invention relates generally to systems and methods for sterilization of powdered material and more particularly to gas sterilization of radiation and heat sensitive powdered materials.

2. Description of the Related Art

Heat and sterilization methods are known that rely on pressure and temperature to eliminate biological contaminants such as bacteria, spores and fungi from a variety of substrates including medical devices, medical compounds and others. Alternately, radiation-based treatments may be used, avoiding some of types of damage to the object to be sterilized that can result from heat and pressure.

In particular, pharmaceutical formulations may have a great deal of sensitivity to damage from heat and pressure, leaving radiation as a primary alternative for sterilization of these compounds. However, radiation having appropriate energies and penetration characteristics for sterilization may also have the effect of damaging the pharmaceutical substrate itself.

### SUMMARY OF THE INVENTION

One aspect of the invention relates to a device configured to sterilize a powder including a device for agitating the powder and a gas supply, configured to apply nitrogen dioxide gas in the presence of humid air to the powder during the agitation.

Another aspect of the invention relates to a method of sterilizing a powder including agitating the powder and exposing the powder to nitrogen dioxide gas in the presence of humid air during the agitating. Particular embodiments of methods in accordance with the present invention include those methods described in the context of the Example below, including each of the methods described in the Tables and associated description.

Yet another aspect of the invention relates to a system configured to control the foregoing device or method including controlling, a rate and/or degree of agitation, a concentration of nitrogen dioxide, a humidity level and a duration of application of the method or operation of the device.

Another aspect of the invention relates to systems, methods and devices of the type described above, but used or performed in a low humidity environment.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic diagram of a device for use in conjunction with a sterilization method in accordance with an embodiment of the invention;

FIG. 2 is a schematic diagram of an alternate embodiment of a tumbling device for use in accordance with an embodiment of the invention;

FIG. 3 is a schematic diagram of a vial lid in accordance with an embodiment of the invention;

FIG. 4 is a chart showing spore population in 100 mg of untreated powder on a log scale where “sample number” corresponds to the untreated samples in chronological order from Example 1;

FIG. 5 is a chart showing spore population in 100 mg of exposed powder on a log scale from Example 1;

FIG. 6 is logarithmic scale of spore population in exposed 100 mg 0.5 mm bead powder samples of varying number of pulse exposures per run;

FIGS. 7a and 7b are perspective views of a system for sterilization in accordance with an embodiment of the present invention;

FIG. 8 is a schematic diagram showing functional interconnections for a system for sterilization in accordance with an embodiment of the present invention;

FIG. 9 is a schematic diagram showing functional interconnections for a sterilant gas delivery subsystem in accordance with an embodiment of the present invention; and

FIG. 10 is a log-linear scale of population against exposure time for an experiment carried out with a dry air diluent.

### DETAILED DESCRIPTION

In view of the issues raised with heat and radiation sterilization approaches, the inventor has determined that gas sterilization may provide good sterilization while mitigating damage to the sterilized substrate. In particular, this approach may be applicable to powdered material.

In an method in accordance with an embodiment of the invention, vials with an amount of powdered biological material, such as a medicament, are placed into a drum within a chamber. One example of a powder of this type is polyvinylpyrrolidone (PVP) which has been used to form drug-containing nano-particles.

The drum is rotatable within the chamber, for example by way of a motor. A sterilizing gas, such as a combination of humid air and NO₂, is provided in the chamber and the vials are rolled during exposure to the gas, ensuring that various portions of the surface area of the powder are exposed to the sterilizing gas. Methods for providing the sterilizing gas to the chamber are described, for example, in U.S. patent application Ser. Nos. 10/585,088, and 11/477,513, herein incorporated by reference.

The sterilizing gas may also be NO₂ without humid air added. In particular, NO₂. In the case that humidity is not applied, diluent gases may be dry air or nitrogen, for example. Alternately, NO₂ alone may be used without any additional diluent gas.

Alternate methods of agitating the powder include dropping the powder through the gas, stirring the powder, vibrating the powder or tumbling the powder during exposure using a different tumbling approach to the one described herein. In principle, a thin layer of powder may be treated without such agitation, however some form of agitation is likely to be useful in ensuring even distribution of sterilizing agent. Agitation may be constant during the treatment, or may alternately be intermittant

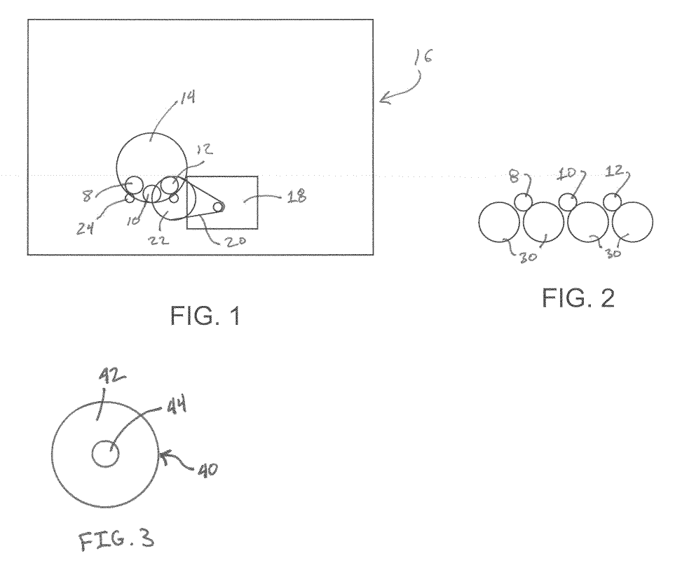

In a particular embodiment, as illustrated in FIG. 1, vials 8, 10, 12 are within a drum 14 that is, in turn, within a sterilization chamber 16. The drum is driven by motor 18, via a drive belt 20 that turns a drive roller 22. An idler wheel 24 supports the drum on the side farthest from the driven wheel. In an embodiment, the motor may be placed outside of the chamber itself, and a drive shaft extending into the chamber may be used to transmit the rotational motion to the interior components. This arrangement can reduce contamination of the inside of the chamber.

Another embodiment may make use of a number of rollers, with one or more vials 8, 10, 12 supported on top of and between adjacent rollers 30, as schematically illustrated in FIG. 2.

In order to allow the sterilizing gas to enter the vials, they should have at least a permeable portion to allow gas and humidity to flow into the vials. In an embodiment, the vials include a breathable cap made from, for example, Tyvek® available from DuPont, or other breathable materials.

In an embodiment, illustrated schematically in FIG. 3, a vial cap 40 has a portion 42 that is made from a permeable material and a portion 44 that is made from a self-healing material, such as rubber for example. Such a configuration may allow access to the vial using a syringe. Thus, a user may inject a fluid into the vial for mixing with the powder and upon mixing, extract the mixed fluid and powder for administration to a patient. Where a self-healing material is used, the insertion of a small syringe will not, in general, result in a breach of the cap such that material can leak or be exposed to other than the material directly injected. In practice, an outer vial cap (not shown) may be additionally included such that at least the permeable portion of the vial cap 40 is covered and sealed except during the sterilization process.

While the method has been herein described as being applied to single-dose vials of material, it may likewise be applied to larger batches of material, prior to further packaging of the material in various useful amounts.

In practice, because the sterilizing gas may contain humidity, clumping of the powder under treatment may occur. As a result, it may be beneficial to include an agitation-aiding agent in the vial with the powder. To this end, glass or other inert beads may be placed in the vial to break up agglomerations. In general, non-spherical beads may provide better anti-clumping performance. For single-dose vials, any agent included in the vial should be both non-reacting with the medical materials, and non-soluble in the solvent (usually sterile water) that will be used to reconstitute the medicament for administration to a patient.

### Example 1

A test to determine whether 100 mg of (PVP) and spore mixture powder could be sterilized using an NO₂ gas sterilization process that incorporates a powder tumbling system. The NO₂ used was a 10% NO₂/90% N₂ mixture.

Vials used in the test had a silicone septum in which a 1.1 cm hole was cut. A 2.2 cm diameter circular Tyvek® pieces was likewise cut and the Tyvek® piece was placed between the cap and the silicone septum ring forming a partially breathable cap on the vials, while maintaining the self-healing characteristic of the silicone septum.

It was found that as the processing of samples matured, the concentration of untreated spores recovered increased, it was believed that this resulted from improved sample handling procedures rather than diminished sterilization functionality. Tumbling the untreated powder and spore mixtures prior to processing had a negative effect on the concentration of spores recovered. There did not seem to be a dependence on humidity for 3.0 mm bead samples. 21 in Hg of humidity added was the least optimal level of humidity added for lethality of 0.5 mm bead samples when two vials were present in the polisher, and the most optimal level of humidity added for lethality of 0.5 mm bead samples when only one vial was present in the polisher. Three pulses had the most lethality at 21 in Hg of humidity added.

The colony forming units (CFU's) recovered from each spore mixture were counted. Multiple plates and dilutions from a given biological indicator (BI) were averaged.

One hundred grams of PVP was placed into each of 15 20 ml vials as shown in Table 1.

TABLE 1
PVP/Spore Mixture Samples Tested in each Run

| | PVP/Spore Mixture |
|---|---|
| Run Number | Types Tested |
| 1 | Two 0.5 mm Beads |
| 2 | Two 3 mm Beads |
| 3 | 0.5 mm Beads and 3 mm |
| | Beads |
| 4 | 0.5 mm Beads and 3 mm |
| | Beads |
| 5 | 0.5 mm Beads and 3 mm |
| | Beads |
| 6 | 0.5 mm Beads and 3 mm |
| | Beads |
| 7 | 0.5 mm Beads and 3 mm |
| | Beads |
| 8 | 0.5 mm Beads |
| 9 | 0.5 mm Beads |
| 10 | 0.5 mm Beads |
| 11 | 0.5 mm Beads |
| 12 | 0.5 mm Beads |
| 13 | 0.5 mm Beads |
| 14 | 0.5 mm Beads |
| 15 | 0.5 mm Beads |

Vials were placed into a cylindrical mesh container. The container was in turn placed into a rock polisher that was configured to spin the container and the vials therein. Conditions within the sterilizer for each of the first two runs are shown in Table 2.

TABLE 2
Run Conditions for 1 and 2

| | | Set Point | | | |
|---|---|---|---|---|---|
| Cycle Step | | Pressure | Dwell Time | | Repeats |
| 1. | Evacuate chamber and pause | 3.1 in Hg | <1 | sec | |
| | (stabilization) | | | | |
| 2. | Add dry air | 29.8 in Hg | <1 | sec | |
| 3. | Repeat initial purge | | | | 1 |
| | (steps 1-2) | | | | |
| 4. | Evacuate chamber and pause | 3.1 in Hg | <1 | sec | |
| 5. | Add NO₂ and pause | 0.4% | <1 | sec | |
| 6. | Add humid air and pause | 25.13 in Hg | <1 | sec | |
| 7. | Add dry air and dwell | 29.8 in Hg | 40 | min | |
| 8. | Evacuate chamber and pause | 3.1 in Hg | <1 | sec | |
| 9. | Add dry air and pause | 29.8 in Hg | <1 | sec | |
| 10. | Repeat final purge (steps 7-8) | | | | 2 |

Run 3 used two vials, one with 100 mg of PVP/spore mixture made with 0.5 mm glass beads (where the 100 mg includes the weight of the beads). The second vial contained 100 mg of PVP/spore mixture with 10 3 mm glass beads (weight of the beads excluded). The test procedure is shown in Table 3.

TABLE 3
Run Conditions for Run 3

| | | Set Point | | | |
|---|---|---|---|---|---|
| Cycle Step | | Pressure | Dwell Time | | Repeats |
| 1. | Evacuate chamber and pause | 3.1 in Hg | <1 | sec | |
| | (stabilization) | | | | |
| 2. | Add dry air | 29.8 in Hg | <1 | sec | |
| 3. | Repeat initial purge | | | | 1 |
| | (steps 1-2) | | | | |
| 4. | Evacuate chamber and pause | 3.1 in Hg | <1 | sec | |
| 5. | Add pre-humidity and dwell | 29.8 in Hg | 10 | min | |
| 6. | Evacuate chamber and pause | 3.1 in Hg | <1 | sec | |
| 7. | Add NO₂ and pause | 0.2% | <1 | sec | |
| 8. | Add humid air and pause | 13.97 in Hg | <1 | sec | |
| 9. | Add dry air | 16.3 in Hg | | | |
| 10. | Add NO₂ and pause | 0.2% | <1 | sec | |
| 11. | Add humid air and pause | 27.47 in Hg | <1 | sec | |
| 12. | Add dry air and dwell | 29.8 in Hg | 10 | min | |
| 13. | Repeat sterilization cycle | | | | 2 |
| | (steps 6-12) | | | | |
| 14. | Evacuate chamber and pause | 3.1 in Hg | <1 | sec | |
| 15. | Add dry air and pause | 29.8 in Hg | <1 | sec | |
| 16. | Repeat final purge (steps 7-8) | | | | 2 |

Runs 4-15 were performed in accordance with the conditions of Table 4 using a single vial of 100 mg PVP/spore mixture made with 0.5 mm glass beads (inclusive of the weight of the beads).

TABLE 4
Run Conditions for Runs 4-15

| | | Set Point | | | |
|---|---|---|---|---|---|
| Cycle Step | | Pressure | Dwell Time | | Repeats |
| 1. | Evacuate chamber and pause | 3.1 in Hg | <1 | sec | |
| | (stabilization) | | | | |
| 2. | Add dry air | 29.8 in Hg | <1 | sec | |
| 3. | Repeat initial purge | | | | 1 |
| | (steps 1-2) | | | | |
| 4. | Evacuate chamber and pause | 3.1 in Hg | <1 | sec | |
| 5. | Add pre-NO₂ and pause | 0.4% | <1 | sec | |
| 6. | Add dry air and dwell | 29.8 in Hg | 10 | min | |
| 7. | Evacuate chamber and pause | 3.1 in Hg | <1 | sec | |
| 8. | Add NO₂ and pause | 0.2% | <1 | sec | |
| 9. | Add humid air and pause | See Table 5 | <1 | sec | |
| 10. | Add dry air | 16.3 in Hg | | | |
| 11. | Add NO₂ and pause | 0.2% | <1 | sec | |
| 12. | Add humid air and pause | See Table 5 | <1 | sec | |
| 13. | Add dry air and dwell | 29.8 in Hg | 10 | min | |
| 14. | Repeat sterilization cycle | | | | See |
| | (steps 7-13) | | | | Table 5 |
| 15. | Evacuate chamber and pause | 3.1 in Hg | <1 | sec | |
| 16. | Add dry air and pause | 29.8 in Hg | <1 | sec | |
| 17. | Repeat final purge (steps 7-8) | | | | 2 |

An overview of the 15 runs is shown in Table 5.

TABLE 5
Overview of Run Conditions

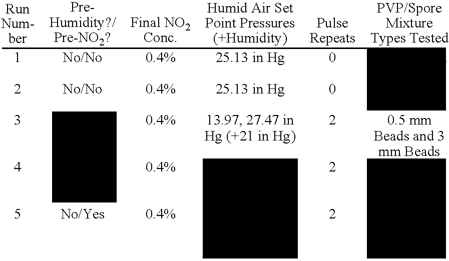

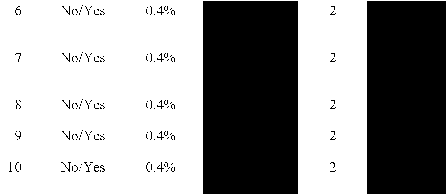

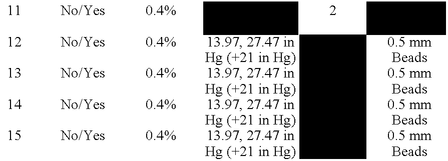

Shaded areas indicates groups of runs with single variable changes

After the runs, the samples were processed and CFUs were counted after approximately 48 hours of incubation at 35° C.-39° C.

Results are shown in Tables 6, 7, and

TABLE 6

| Run Number | | Colony | Dilution | CFU per | Average per | Standard |
|---|---|---|---|---|---|---|
| and Name | Bead Size | Forming Units | Factor | 100 mg | 100 mg | Deviation |
| 1 | 0.5 mm | 129 | 100 | 1.3 × 10⁴ | 2.4 × 10⁴ | 1.8 × 10⁴ |
| NX1080422A | | 123 | 100 | 1.2 × 10⁴ | | |
| | | 2 | 10000 | 2.0 × 10⁴ | | |
| | | 5 | 10000 | 5.0 × 10⁴ | | |
| | 0.5 mm | 270 | 100 | 2.7 × 10⁴ | 6.2 × 10⁴ | 5.9 × 10⁴ |
| | | 307 | 100 | 3.1 × 10⁴ | | |
| | | 4 | 10000 | 4.0 × 10⁴ | | |
| | | 15 | 10000 | 1.5 × 10⁵ | | |
| UT₁* | 0.5 mm | 1186 | 100 | 1.2 × 10⁵ | 2.3 × 10⁵ | 1.5 × 10⁵ |
| | | 1108 | 100 | 1.1 × 10⁵ | | |
| | | 42 | 10000 | 4.2 × 10⁵ | | |
| | | 27 | 10000 | 2.7 × 10⁵ | | |
| 2 | 3.0 mm | >2000 | 100 | >2.0 × 10⁵ | >2.0 × 10⁵ | — |
| NX1080423A | 5 beads | >2000 | 100 | >2.0 × 10⁵ | | |
| | 3.0 mm | >2000 | 100 | >2.0 × 10⁵ | >2.0 × 10⁵ | — |
| | 10 beads | >2000 | 100 | >2.0 × 10⁵ | | |
| UT₂* | 3.0 mm | 689 | 1000 | 6.9 × 10⁵ | 6.4 × 10⁵ | 7.3 × 10⁴ |
| | | 586 | 1000 | 5.9 × 10⁵ | | |

*Untreated samples were not tumbled as exposed samples were.

TABLE 7

| Run Number | | Colony | Dilution | CFU per | Average per | Standard |
|---|---|---|---|---|---|---|
| and Name | Bead Size | Forming Units | Factor | 100 mg | 100 mg | Deviation |
| 3 | 0.5 mm | 26 | 100 | 2.6 × 10³ | 3.4 × 10³ | 3.8 × 10³ |
| NX1080424A | | 11 | 100 | 1.1 × 10³ | | |
| | | 1 | 1000 | 1.0 × 10³ | | |
| | | 9 | 1000 | 9.0 × 10³ | | |
| | 3.0 mm | 680 | 100 | 6.8 × 10⁴ | 8.6 × 10⁴ | 2.6 × 10⁴ |
| | | 680 | 100 | 6.8 × 10⁴ | | |
| | | 85 | 1000 | 8.5 × 10⁴ | | |
| | | 123 | 1000 | 1.2 × 10⁵ | | |
| 4 | 0.5 mm | 11 | 100 | 1.1 × 10³ | 1.8 × 10³ | 9.3 × 10² |
| NX1080424B | | 19 | 100 | 1.9 × 10³ | | |
| | | 1 | 1000 | 1.0 × 10³ | | |
| | | 3 | 1000 | 3.0 × 10³ | | |
| | 3.0 mm | 400 | 100 | 4.0 × 10⁴ | 4.0 × 10⁴ | 9.6 × 10³ |
| | | 324 | 100 | 3.2 × 10⁴ | | |
| | | 54 | 1000 | 5.4 × 10⁴ | | |
| | | 35 | 1000 | 3.5 × 10⁵ | | |
| UT₃₊₄* | 0.5 mm | TNTC | 100 | — | 6.7 × 10⁵ | 6.5 × 10⁵ |
| | | TNTC | 100 | — | | |
| | | 1124 | 1000 | 1.1 × 10⁶ | | |
| | | 207 | 1000 | 2.1 × 10⁵ | | |
| | 3.0 mm | TNTC | 100 | — | 5.1 × 10⁵ | — |
| | | TNTC | 100 | — | | |
| | | 510 | 1000 | 5.1 × 10⁵ | | |
| | | Contaminated | 1000 | — | | |

*Untreated samples were not tumbled as exposed samples were.

TABLE 8

| Run Number | | Colony | Dilution | CFU per | Average per | Standard |
|---|---|---|---|---|---|---|
| and Name | Bead Size | Forming Units | Factor | 100 mg | 100 mg | Deviation |
| 5 | 0.5 mm | 38 | 10 | 3.8 × 10² | 3.5 × 10² | 1.2 × 10² |
| NX1080429A | | 23 | 10 | 2.3 × 10² | | |
| | | 3 | 100 | 3.0 × 10² | | |
| | | 5 | 100 | 5.0 × 10² | | |
| | 3.0 mm | 356 | 100 | 3.6 × 10⁴ | 3.6 × 10⁴ | 3.0 × 10² |
| | | 354 | 100 | 3.5 × 10⁴ | | |
| | | 36 | 1000 | 3.6 × 10⁴ | | |
| | | 36 | 1000 | 3.6 × 10⁴ | | |
| 6 | 0.5 mm | 7 | 10 | 7.0 × 10¹ | 1.1 × 10² | 7.5 × 10¹ |
| NX1080429B | | 7 | 10 | 7.0 × 10¹ | | |
| | | 2 | 100 | 2.0 × 10² | | |
| | | Contaminated | 100 | — | | |
| | 3.0 mm | 157 | 100 | 1.6 × 10⁴ | 1.9 × 10⁴ | 5.3 × 10³ |
| | | 162 | 100 | 1.6 × 10⁴ | | |
| | | 18 | 1000 | 1.8 × 10⁴ | | |
| | | 27 | 1000 | 2.7 × 10⁴ | | |
| 7 | 0.5 mm | 8 | 10 | 8.0 × 10¹ | 1.3 × 10² | 5.3 × 10¹ |
| NX1080429C | | 12 | 10 | 1.2 × 10² | | |
| | | 1 | 100 | 1.0 × 10² | | |
| | | 2 | 100 | 2.0 × 10² | | |
| | 3.0 mm | 213 | 100 | 2.1 × 10⁴ | 2.3 × 10⁴ | 4.1 × 10³ |
| | | 196 | 100 | 2.0 × 10⁴ | | |
| | | 29 | 1000 | 2.9 × 10⁴ | | |
| | | 23 | 1000 | 2.3 × 10⁴ | | |
| UT₅₊₆₊₇* | 0.5 mm | 141 | 10000 | 1.4 × 10⁶ | 1.5 × 10⁶ | 1.4 × 10⁵ |
| | | 161 | 10000 | 1.6 × 10⁶ | | |
| | 3.0 mm | 511 | 10000 | 5.1 × 10⁶ | 5.4 × 10⁶ | 3.4 × 10⁵ |
| | | 559 | 10000 | 5.6 × 10⁶ | | |

*Untreated samples were not tumbled as exposed samples were.

TABLE 9

| Run Number | | Colony | Dilution | CFU per | Average per | Standard |
|---|---|---|---|---|---|---|
| and Name | Bead Size | Forming Units | Factor | 100 mg | 100 mg | Deviation |
| 8 | 0.5 mm | 30 | 2 | 6.0 × 10¹ | 1.8 × 10² | 5.6 × 10¹ |
| NX1080501B | | 23 | 10 | 2.3 × 10² | | |
| | | 17 | 10 | 1.7 × 10² | | |
| | | 1 | 100 | 1.0 × 10² | | |
| | | 2 | 100 | 2.0 × 10² | | |
| 9 | 0.5 mm | 51 | 2 | 1.0 × 10² | 3.3 × 10¹ | 4.7 × 10¹ |
| NX1080501C | | 3 | 10 | 3.0 × 10¹ | | |
| | | 10 | 10 | 1.0 × 10² | | |
| | | 0 | 100 | 0.0 × 10⁰ | | |
| | | 0 | 100 | 0.0 × 10⁰ | | |
| 10 | 0.5 mm | Mislabeled | 2 | — | 5.0 × 10⁰ | 5.8 × 10⁰ |
| NX1080501D | | 1 | 10 | 1.0 × 10¹ | | |
| | | 1 | 10 | 1.0 × 10¹ | | |
| | | 0 | 100 | 0.0 × 10⁰ | | |
| | | 0 | 100 | 0.0 × 10⁰ | | |
| 11 | 0.5 mm | Mislabeled | 2 | — | 1.3 × 10² | 9.2 × 10¹ |
| NX1080501E | | 19 | 10 | 1.9 × 10² | | |
| | | 12 | 10 | 1.2 × 10² | | |
| | | 0 | 100 | 0.0 × 10⁰ | | |
| | | 2 | 100 | 2.0 × 10² | | |
| UT₈₊₉₊₁₀₊₁₁ | 0.5 mm | 358 | 100 | 3.6 × 10⁴ | 3.4 × 10⁴ | 2.5 × 10³ |
| | | 323 | 100 | 3.2 × 10⁴ | | |
| | | 40 | 1000 | 4.0 × 10⁴ | | |
| | | 48 | 1000 | 4.8 × 10⁴ | | |
| | | 3 | 10000 | 3.0 × 10⁴ | | |
| | | 4 | 10000 | 4.0 × 10⁴ | | |

TABLE 10

| Run Number | | Colony | Dilution | CFU per | Average per | Standard |
|---|---|---|---|---|---|---|
| and Name | Bead Size | Forming Units | Factor | 100 mg | 100 mg | Deviation |
| 12 | 0.5 mm | 14 | 2 | 2.8 × 10¹ | 2.2 × 10¹ | 2.0 × 10¹ |
| NX1080505A | | 4 | 10 | 4.0 × 10¹ | | |
| | | 4 | 10 | 4.0 × 10¹ | | |
| | | 0 | 100 | 0.0 × 10⁰ | | |
| | | 0 | 100 | 0.0 × 10⁰ | | |
| 13 | 0.5 mm | 0 | 2 | 0.0 × 10⁰ | 2.0 × 10⁰ | 4.5 × 10⁰ |
| NX1080505B | | 1 | 10 | 1.0 × 10¹ | | |
| | | 0 | 10 | 0.0 × 10⁰ | | |
| | | 0 | 100 | 0.0 × 10⁰ | | |
| | | 0 | 100 | 0.0 × 10⁰ | | |
| 14 | 0.5 mm | 1 | 2 | 2.0 × 10⁰ | 4.0 × 10⁻¹ | 8.9 × 10⁻¹ |
| NX1080505C | | 0 | 10 | 0.0 × 10⁰ | | |
| | | 0 | 10 | 0.0 × 10⁰ | | |
| | | 0 | 100 | 0.0 × 10⁰ | | |
| | | 0 | 100 | 0.0 × 10⁰ | | |
| 15 | 0.5 mm | 0 | 2 | 0.0 × 10⁰ | 8.0 × 10⁰ | 1.1 × 10¹ |
| NX1080505D | | 2 | 10 | 2.0 × 10¹ | | |
| | | 2 | 10 | 2.0 × 10¹ | | |
| | | 0 | 100 | 0.0 × 10⁰ | | |
| | | 0 | 100 | 0.0 × 10⁰ | | |
| UT₁₂₊₁₃₊₁₄₊₁₅ | 0.5 mm | 354 | 10000 | 3.5 × 10⁴ | 3.9 × 10⁴ | 7.1 × 10³ |
| | | 358 | 10000 | 3.6 × 10⁴ | | |
| | | 36 | 10000 | 3.6 × 10⁴ | | |
| | | 50 | 10000 | 5.0 × 10⁴ | | |

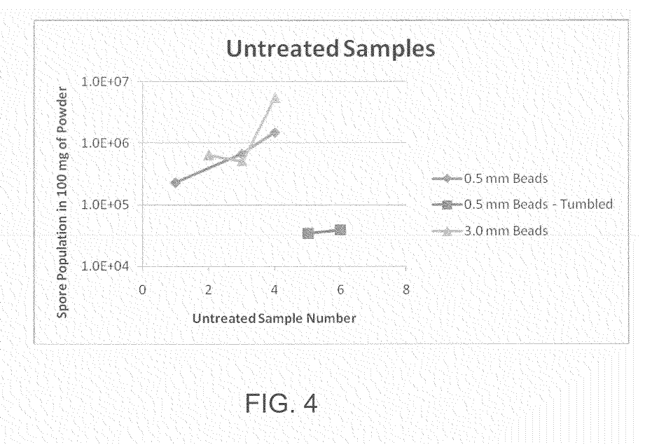

As shown in FIG. 4, there is an increase in the recovery of untreated samples for both 0.5 mm and 3.0 mm beads as the recovery process matured. It is expected that the 3.0 mm bead samples will have a larger number of spores as the weight of the 0.5 mm beads included in the total weight of the 0.5 mm bead samples, while the 3.0 mm bead samples did not include bead weight. The average for the untreated 3.0 mm bead samples was 2.2×10⁶ spores/100 mg, while the untreated 0.5 mm bead samples had an average 8.0×10⁵ spores/100 mg. However, there is more than a log decrease seen for those 0.5 mm bead samples that were tumbled prior to processing. The average of those samples was 3.7×10⁴ spores/100 mg. Part of this decrease may be attributable to some powder not being dissolved into the water when added. The powder may have been stuck to the lid of the vial and not dissolved, or could have aggregated in the vial and was not given sufficient time to dissolve.

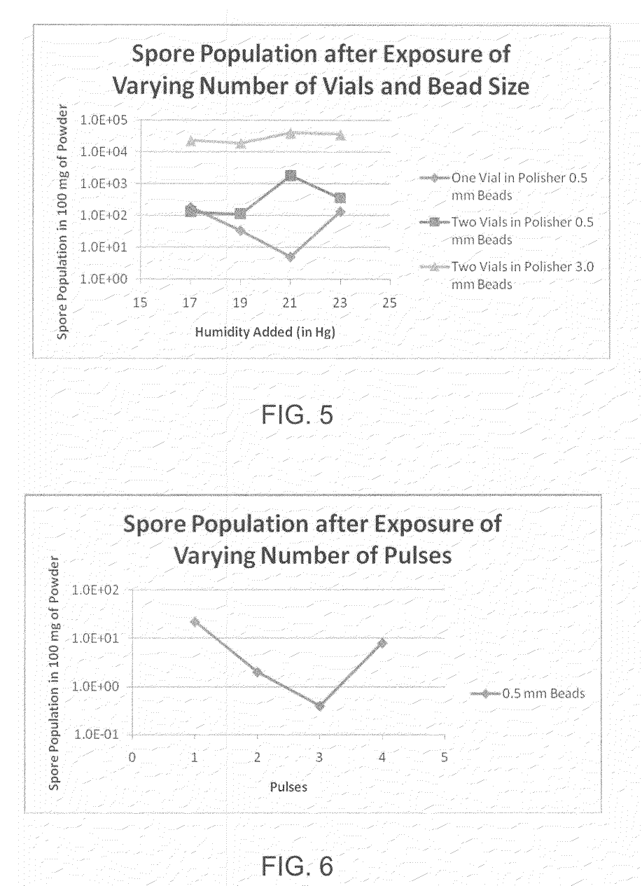

As shown in FIG. 5, the spore population of 100 mg of 3.0 mm bead treated powder samples seems constant, between 2.0×10⁴ to 4.0×10⁴ spores, from the addition of 17 in Hg through 23 in Hg of humidity. However, when two vials were present, the 0.5 mm bead samples seemed to have the least amount of lethality at 21 in Hg of humidity added, yielding a concentration of 1.8×10³ spores/100 mg. This concentration decreased as the humidity amount was increased or decreased, 1.3×10² spores/100 mg and 3.5×10² spores/100 mg for 17 in Hg and 23 in Hg of humidity added, respectively. Conversely, when one vial was present within the polisher, the graph has an inverse shape. It is at 21 in Hg of humidity added that the greatest lethality existed, leaving only 5.0×100 spores/100 mg. This data is more consistent with the theory that too little humidity will not produce enough lethality, while too much humidity will cause clumping of the powder and protect spores from the sterilant.

The data seen in FIG. 6 is also consistent with the theory that there is an optimal humidity level, and that too much or not enough will lead to a decrease in lethality. As the number of pulses increases, the amount of humidity that the powder and spore mixture is exposed to is increased. The optimal number of pulses with 21 in Hg of humidity added seems to be three, yielding a final concentration of 4.0×10⁻¹ spores/100 mg.

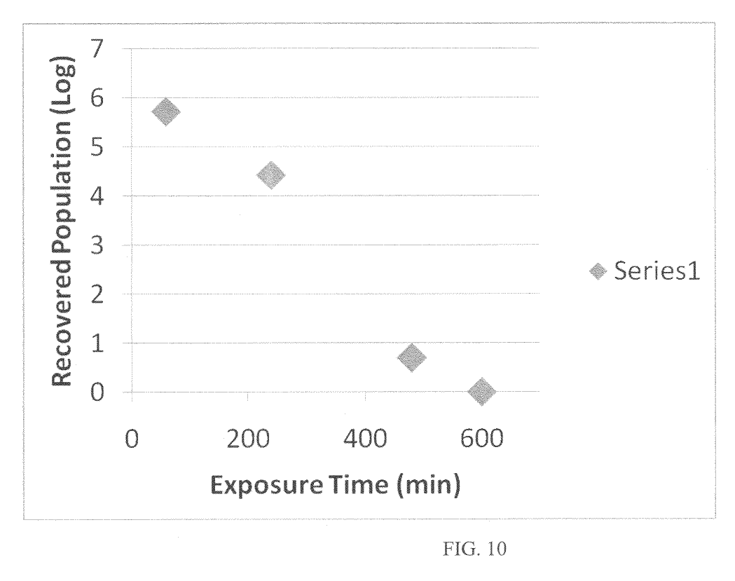

On the other hand, additional research has shown that using a fixed concentration of NO₂ gas (10 mg/l) and exposure time ranging from 60 minutes (1 hour) to 600 minutes (10 hours) resulted in acceptable lethality. Within this range of exposure durations, the dry conditions resulted in measureable inactivation kinetics that follow a log-linear response, which is shown in FIG. 10.

In an embodiment, a low concentration (<21 mg/L) of nitrogen dioxide gas in the presence of air and water vapor is delivered to a sterilization chamber. In particular embodiments, concentrations of about 5 to 10 mg/L are used. As described in greater detail below, the process may be performed at or near room temperature and entails evacuating air from the chamber, introducing the sterilant gas, and adding humidified (or dry) air to a selected pressure. Depending on the physical characteristics and/or packaging of the item to be sterilized, the sequence of vacuum→sterilant injection→humid air injection, may be repeated several times or the sequence changed. Furthermore, additional sequence steps of dry air injection and dwell may be included in one or all iterations of the sterilizing sequence. At the ordinary operating temperatures and pressures of the process, the NO₂ remains in the gas phase and acts as an ideal gas.

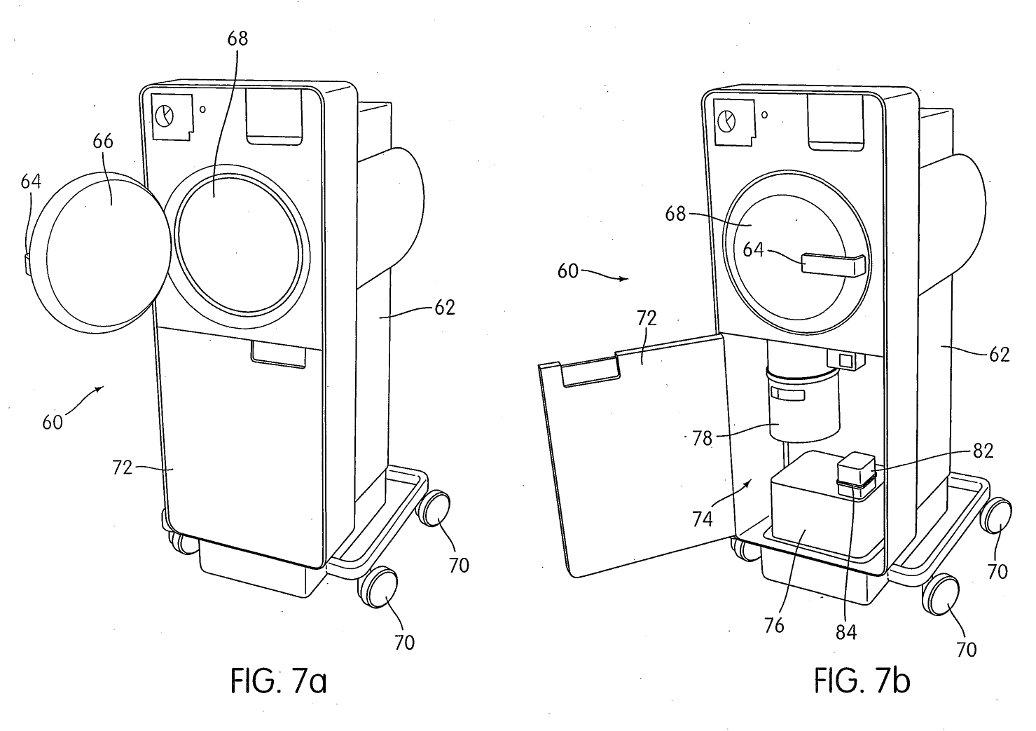

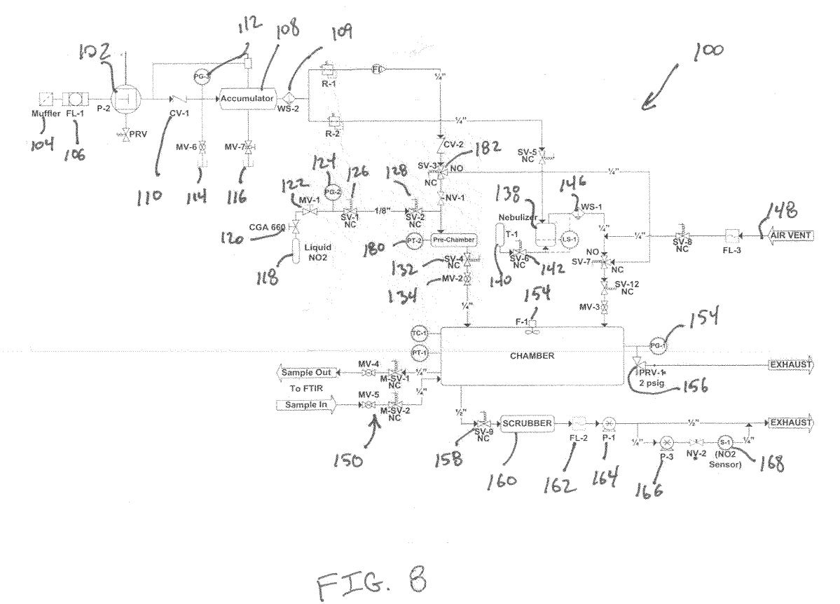

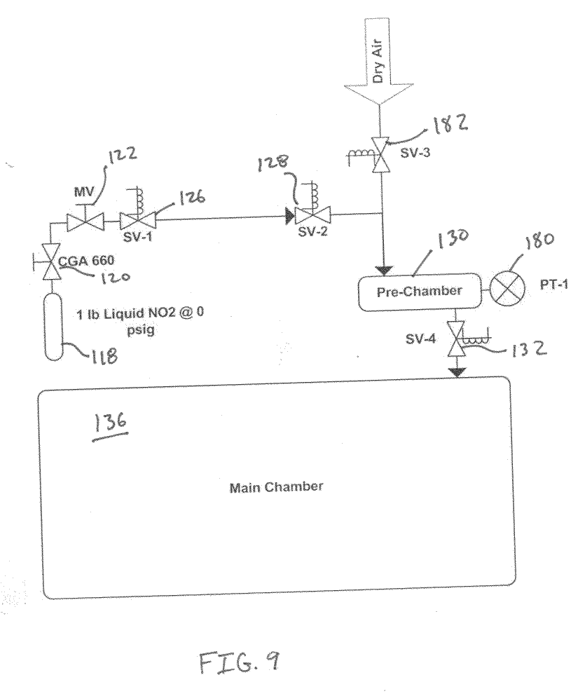

An embodiment of a sterilizer that uses NO₂ sterilizing gas is illustrated generally in FIGS. 7a and 7b. The sterilizer 60 includes a housing 62. In an embodiment, the housing 62 is sized such that a handle 64 for a door 66 for the sterilizing chamber 68 is at a height suited to use by an average standing user, for example, about 42″. The overall height of such a system may be about 5 feet and the width, approximately 20″. As shown, the housing 62 may optionally be supported on a set of wheels 70, to allow for easy portability of the sterilizer 60.

A second door 72 is located in a lower portion of the housing 62 and allows access to serviceable portions of the sterilizer 60. In particular, consumables may be stored in the service area 74. In the embodiment shown, a sterilant gas module 76 and a scrubber 78 are located in the service area, along with a reservoir 80 for storing water to be used by a humidification system, as described below. The sterilant gas module includes a door 82 having a hinge 84 allowing it to be opened for access to replace a sterilant gas source (not shown), as described in greater detail below.

FIG. 8 is a schematic process and instrumentation diagram of an embodiment of a sterilizer 100 in accordance with the present invention. A first portion of the sterilizer 100 is a source of air to be added to the nitrogen dioxide gas in the chamber. A compressor 102 compresses air from the ambient environment. Prior to compression, the ambient air passes through a muffler 104 and a filter 106. The filter 106 reduces dust and other particulate impurities that are generally undesirable both for the compressor and the downstream use of the compressed air. Furthermore, the filter 106 may advantageously be designed to remove microbes from the air stream such that the air delivered to the sterilizer, and in particular to the humidification system, is substantially pathogen free. As will be appreciated, other sources of air may be substituted. For example, air may be provided by air tanks or a fixed air supply system that provides pressurized air to the room in which the sterilizer is housed.

As shown, the air is supplied from the compressor 102 to an accumulator 108 via a control valve 110. In the illustrated embodiment, pressure in the accumulator 108 is controlled via a feedback loop to the control valve 110 using a pressure gage 112. Manual valves 114, 116 are optionally provided to allow pressure relief and water drain from the accumulator 108 respectively. A water separator 109 may be included to ensure that liquid water does not enter the air stream on the downstream side of the accumulator.

Nitrogen dioxide is provided to the system from a liquid supply tank 118. A manual valve 120 and a valve 122 control flow from the supply tank 118. A pressure gage 124 allows monitoring of pressure in the lines and a pair of solenoid valves 126, 128 control flow into a pre-chamber 130. Another pair of valves 132, 134 control flow from the pre-chamber 130 to the sterilization chamber 136. More detail of the operation of the NO₂ delivery sub-system is discussed below.

A sub-system for providing humidity to the sterilization chamber 136 begins with a Collison nebulizer 138 that produces aerosolized water in air to be provided to the sterilization chamber 136. The air for this process is provided by the accumulator 108, similarly to the air used in the pre-chamber 130. Water for humidification is stored in the reservoir 140, and a solenoid valve 142 controls water flow from the reservoir 140 into the nebulizer 138. A level sensor 144 monitors the water level in the nebulizer 138 and controls the opening of the solenoid valve 142. As the pressurized air enters the nebulizer, it generates a sonic velocity air jet in water held in the nebulizer. The air jet aspirates the water, forming small droplets which then vaporize. A water separator 146 prevents liquid water from entering the sterilization chamber 136 while allowing the humid air to pass through. An air vent 148 provides a vent pathway from the nebulizer allowing the water to flow from the reservoir 140 to the nebulizer 138. Suitable valves control the entry of the humidified air to the sterilization chamber 136.

As illustrated, the sterilization chamber 136 includes access via a set of valves 150 so that samples of the chamber atmosphere may be taken and analyzed. Analysis may be, for example, by an FTIR, UV spectrophotometric, or other appropriate spectrometry system, not shown. Access for analysis has particular relevance to a test platform, and may be unnecessary in practice when the sterilizer is used in a production environment.

The sterilization chamber 136 may include a fan 152 that helps to circulate gases in the chamber. Circulation helps to ensure both that the sterilant gas is well mixed with the humidified air, and that objects to be sterilized are well exposed to the sterilant gas.

A pressure gage 154 and pressure relief valve 156 may be provided to control pressures in the sterilization chamber 136. As will be appreciated, in the case that exhaust from the pressure relief pathway contains nitrogen dioxide, it should be controlled or processed to avoid contamination of the work area.

The primary exhaust pathway proceeds through a solenoid valve 158 to a scrubber 160, designed to eliminate and/or capture nitrogen dioxide before the exhaust reaches the environment. A filter 162 removes particulates from the exhaust. Pump 164 pushes scrubbed exhaust out of the system. Another pump 166 provides a flow through an NO₂ sensor 168 for monitoring NO₂ content of the exhaust gases. Should the NO₂ levels exceed a selected threshold, solenoid valve 158 can be closed to ensure that NO₂ is not released into the environment.

FIG. 9 illustrates an embodiment of a sterilant delivery system similar in configuration to the sterilant delivery sub-system of FIG. 7. A tank 118 containing liquid NO₂ acts as the source of sterilant gas. A manual valve 120 provides a flow of gas from the tank 118. A manual valve 122 provides a secondary control over flow from the tank. A pair of solenoid valves 126, 128 are actuatable to allow flow from the valve to the sterilizing system. As illustrated, there are four separate valves that ultimately control flow from the tank 118. As will be appreciated, other valve arrangements are possible, and redundancy may be reduced or eliminated, as desired.

During use, sterilant gas is allowed to flow from the final solenoid valve 128 into a pre-chamber 130, where it expands and the dosage may be measured. As shown, the pre-chamber 130 includes a pressure transducer 180 that allows measurement of a total pressure which may be translated into dosage, given appropriate knowledge of the size of the chamber and optionally, temperature data derived from a temperature sensor, not shown. A solenoid valve 132 controls flow into the sterilizing chamber 136. An additional solenoid valve 182 controls flow of dry air into the pre-chamber.

In one method of operating the illustrated embodiment, the chamber 136 and pre-chamber 130 are initially at low pressure, for example, they may be evacuated using appropriate vacuum pumps (for example, the pump 164 in the exhaust pathway illustrated in FIG. 8). In an embodiment, an evacuation cycle is repeated prior to injection of the sterilant gas. As an example, the chambers may be evacuated, re-filled with air, and then evacuated again prior to initiating the sterilant gas sequence.

In order to begin delivery of sterilant gas, valve 128 is closed and 132 is opened, while valve 182 is held closed, equalizing the pressure in the chamber 136 and pre-chamber 130 at a low pressure. Valve 132 is closed, isolating the pre-chamber 130 from the sterilizing chamber 136. Valves 126 and 128 are then opened (valve 122 and manual valve 120 having been already opened) and gas that has boiled off of the liquid NO₂ supply is allowed to enter the pre-chamber 130. The pressure transducer 180 may be used in a feedback arrangement to control solenoid valve 126 such that a selected total amount of NO₂ is collected in the pre-chamber 130.

As will be appreciated, if volume of the pre-chamber 130, pressure and temperature are known, for example via measurements using the pressure transducer 180 and a temperature gage (not shown), the total amount of NO₂ in the pre-chamber 130 may be calculated. By way of example, an operating pressure of 10-20 in Hg may be generated in order to provide an approximately 0.5 gram dose of sterilant to a sterilization chamber 136 having a volume of about 60 liters. In this approach, a concentration of about 0.5% sterilant gas is produced in the sterilization chamber 136.

After the pre-chamber 130 is pressurized, the valves 126 and 128 are closed, isolating the pre-chamber 130 from the gas source. Valve 132 is opened, allowing the gas from the pre-chamber 130 to pass into the sterilizing chamber 136. Valve 182 is opened to allow dry air to enter into the sterilizing chamber 136, and to push any remaining sterilant gas out of the pre-chamber 130 and into the sterilizing chamber 136. Finally, valves 182 and 132 are closed, isolating the sterilizing chamber from the other portions of the system.

In an embodiment, the additional chamber, which may be the pre-chamber, or an additional chamber, is used to circulate the sterilizing gas into and out of the sterilizing chamber. For example, a pre-chamber or co-chamber of sufficient size may be used for recycling the sterilizing atmosphere. In this case, the sterilization cycle may be initiated in the manner described with respect to the other embodiments. The pre-chamber or co-chamber can be opened to the sterilizing chamber, via a circuit that may include a pump for driving the gas from the sterilizing chamber to the alternative chamber volume. Then, the gas can be re-introduced to the sterilizing chamber. This re-introduction may occur one time, more than one time, or the gases may be continuously transferred from one chamber to the other. The inventors have determined that repeated exposure cycles may be more effective for sterilization than a longer dwell, single exposure cycle. The removal and re-introduction of the sterilant gas will achieve the same ends as the repeated exposure cycles. The concentration of sterilant or the humidity of the gases being transferred between the two chambers may be adjusted to maintain lethal exposure conditions.

As will be appreciated, other configurations and methods may be used to provide the sterilant gas to the sterilizing chamber 136. For example, a gas source may be used in place of the liquid source. The source may be a single use source, or multiple use source as shown. Other valving arrangements and control sequences may replace those described herein. Liquid or solid source material may be provided directly to the sterilizing chamber 136, without first being converted to a gas. As an example, a material that is known to produce NO (which may be converted to NO₂ in use) is described in U.S. patent application Ser. No. 11/052,745, filed Sep. 15, 2005, and herein incorporated by reference in its entirety. Likewise, gas may be delivered at varying concentrations to the chamber. That is, while the described method provides a high concentration sterilant gas to the chamber, there may be greater or lesser degrees of mixing with air prior to delivery.

In an embodiment, a non-reactive gas or gas mixture rather than dry air is added to dilute the sterilant gas. For example, N₂ gas may be used in place of air. In this approach, the N₂ gas may be used dry, humidified prior to adding to the sterilization chamber 136, or may alternately be humidified in the sterilization chamber 136, as with the embodiments using air.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of sterilizing a powdered material comprising:
placing the powdered material in a sterilization chamber;
exposing the powder to nitrogen dioxide gas in the sterilization chamber at substantially room temperature, wherein the exposing includes controlling a concentration of nitrogen dioxide gas to which the powder is exposed; and
during the exposing, agitating the powder, wherein the agitating the powder includes controlling an amount and duration of the agitating.

2. A method as in claim 1, wherein the exposing further comprises controlling an amount of humidity in the sterilization chamber.

3. A method as in claim 1, wherein the controlling a concentration of nitrogen dioxide gas includes controlling an amount of diluent gas added to the sterilization chamber.

4. A method as in claim 4, wherein the diluent gas comprises a gas selected from the group consisting of: dry air, humidified air, water vapor, nitrogen gas and combinations thereof.

5. A method as in claim 1, wherein the powder is in a container, and the agitating comprises rotating the container during the exposing.

6. A method as in claim 5, wherein the container comprises a vial having at least one gas-permeable portion and at least one gas-impermeable portion.

7. A method as in claim 6, wherein the vial is rotated by at least one rotating roller in contact with the vial.

8. A vial for use in a gas-exposure process, comprising:
a substantially tubular body member having an opening at one end;
a seal, the seal configured to close the opening and including a self-healing portion and a gas-permeable portion, the gas-permeable portion being permeable to a gas used in the gas exposure process.

9. A vial as in claim 8, wherein the gas-permeable portion is permeable to at least NO₂.

10. A vial as in claim 9, wherein the gas-permeable portion is further permeable to at least one gas selected from the group consisting of: dry air, nitrogen gas, humidified air, water vapor and combinations thereof.

11. A vial as in claim 8, wherein the gas-permeable portion comprises a nonwoven spunbonded olefin fiber and the self healing portion comprises a rubber material.

12. A device for sterilizing a powder comprising:
a process chamber;
an agitation device, configured and arranged to agitate the powder within the process chamber during the sterilizing; and
a gas supply, configured and arranged to provide a nitrogen dioxide gas to the process chamber in a controlled amount.

13. A device as in claim 12, wherein the gas supply is further configured and arranged to provide humid air to the chamber in a controlled amount.

14. A device as in claim 12, wherein the gas supply further comprises:
a source of liquid nitrogen dioxide;
a sterilization pre-chamber, in fluid communication with the source of liquid nitrogen dioxide such that gaseous liquid nitrogen dioxide produced by vaporization of the liquid nitrogen dioxide may be contained in the sterilization pre-chamber, the sterilization pre-chamber further being in fluid communication with the process chamber such that gaseous liquid nitrogen dioxide may be controllably delivered into the process chamber.

15. A device as in claim 14, further comprising a source of humidity in fluid communication with the process chamber such that a humidity of the process chamber may be controlled.

16. A device as in claim 14, wherein the agitation device comprises a roller, operable to rotate a container containing the powder during use.
